(19) United States
(12) Patent Application Publication
(10) Pub. No.: US 2003/0078400 A1
(43) Pub. Date: Apr. 24, 2003

(54) Compounds for the modulation of kinase associated signal transduction

(75) Inventors: Morris Laster, Jerusalem (IL); Dror Tobi, Beit Shemesh (IL)

Correspondence Address: BROWDY AND NEIMARK, P.L.L.C., 624 NINTH STREET, NW, SUITE 300, WASHINGTON, DC, 20001-5303, US

(73) Assignee: Keryx Biopharmaceuticals, Inc., East Cambridge, MA (US)

(21) Appl. No.: 10/205,302

(22) Filed: Jul. 26, 2002

Related U.S. Application Data
(60) Provisional application No. 60/307,592, filed on Jul. 26, 2001.

(51) Int. Cl. C07H 21/02

(52) U.S. Cl. 536/23.1

(57) ABSTRACT

The invention concerns a hardware-software system comprising information on the three-dimensional structure and nature of certain regions in protein kinases. The invention also concerns methods for screening for or synthesizing candidate chemical compounds for regulating kinase activity based on this information.

### REFERENCE TO TABLE ON COMPACT DISC

Table 1 (which has more than 50 pages of text) is presented as two files on a read-only compact disc, which disc is attached hereto along with a duplicate thereof. The only files on each disc that which contains Table 1 and are titled COORDINATES_OUT_PART1.doc and COORDINATES_OUT_PART2.doc, which files were created on Jul. 25, 2002, and have a size of 3,347 KB and 3,311 KB, respectively. The file titled COORDINATES_OUT_PART2.doc is a continuation of the file marked COORDINATES_OUT_PART1.doc and both files together form all of Table 1. In accordance with 37 CFR 1.52(e)(5), applicants hereby incorporate by reference all of the material on the above-identified compact discs.

### FIELD OF THE INVENTION

The present invention concerns a hardware-software system comprising encoded data storage medium capable of displaying the atomic coordinates of specific regions within a protein. The present invention further concerns methods using said atomic coordinates, for synthesizing, evaluating or screening for molecules, which can be used for the modulation of enzymatic activities.

### BACKGROUND OF THE INVENTION

The eukaryotic protein kinase super family is composed of enzymes that use the gamma phosphate of ATP or GTP to specifically phophorylate serine, threonine or tyrosine residues of intracellular proteins. Along with the cycle catalyzed by G-proteins and their associated factors, protein phosphorylation cycles are the primary means for rapidly switching the activities of cellular proteins from one state to the other. Three major classes of inputs modulate protein phosphorylation cycles: external signals, cell cycle checkpoints and environmental or nutritional stress.

Many of the kinases are involved in signal transduction in multicellular organisms in a variety of cellular events. Kinases were found to be involved in a wide range of physiological phenomena including: cellular proliferation, cellular differentiation, ontogenesis, immune response and inflammatory responses.

Non-normal kinase activity has been found to be involved in proliferation-based diseases such as cancer; in non-malignant proliferative diseases such as arteriosclerosis and psoriasis; and in inflammatory responses such as septic shock Agents that can modulate (by increasing or decreasing) the activity of protein kinases have a potential for the treatment of a wide variety of diseases and disorders including: cancel; autoimmune diseases, inflammation, injury, metabolic disorders, genetic disorders and the like.

PKs are known to have homologous “kinase domains” or “catalytic domains” which are responsible to the phosphorylation activity. Based on a comparison of a large number of protein kinases, it is now known that the kinase domain of protein kinases can be divided into twelve subdomains. These are regions that are generally uninterrupted by large amino acid insertions and contain characteristic patterns of conserved residues (Hanks and Hunter; “The Eukaryotic Protein Kinase Superfamily”, in Hardie and Hanks ed., The Protein Kinase Facts Book, Volume I, Academic Press, Chapter 2, 1995). These subdomains are referred to as Subdomain I through Subdomain XII.

Due to the high degree of homology found in the subdomains of different protein kinases, the amino acid sequences of the domains of different PKs can be aligned. Frequently, the alignment is with reference to the prototypical protein kinase PKA-C_{α}, as known in the art. Currently, the catalytic domains of a large number of protein kinases have been aligned and tables showing these alignments are available from various published sources, such as, for example, in the article by Hanks and Quinn in Methods of Enzymology 200, 38-62 (1991) or in the PKR Web Site: WWW.sdsc.edu/kinases.

U.S. Pat. No. 6,174,993, WO98/53051 (corresponding to pending U.S. application Ser. No. 08/861,153), WO 00/118895 (corresponding to pending U.S. 09/161,094), U.S. pending application Ser. No. 09/458,491 and U.S. Ser. No. 09/734,520 (all incorporated herein by reference) concern small, previously undisclosed, regions of various protein kinases with a high substrate specificity. Short peptides derived from these regions modulate kinase activities, as determined by the modulation of cellular activity in various in vivo and in vitro models. Without wishing to be bound by theory it is assumed that the short peptides disclosed in these applications, which mimic some of the catalytic domains of the kinase, bind to other cellular components with which the kinase interacted (such as substrates, other kinases, other phosphatases) and thus either mimic the kinase activity, or alternatively inhibits the interaction of the kinase and the cellular components thus inhibiting kinase activity.

U.S. Pat. No. 6,174,993 and U.S. application Ser. No. 08/861,153 discloses a domain termed the HJ-loop. The “HJ-loop” referred to herein is found within the kinase domain of ser/thr protein kinases between the middle of Subdomain IX and the middle of Subdomain X. Because of the high degree of homology found in the subdomains of different protein kinases, the amino acid sequences of the domains of different ser/thr protein kinases can be aligned. Thus, the HJ-loop of a protein kinases can be defined by reference to the amino acid sequence of a prototypical protein kinase, for example PKA-C_{α}, and can be said to correspond to a contiguous sequence of about twenty amino acid residues found between about amino acid 229 and 248 of PKA-C_{α}

A second definition of the HJ loop of protein kinases, which is complementary to the definition provided in the proceeding paragraph, can be made by reference to the secondary structure of the kinase domain of protein kinases. The kinase domain of protein kinases has been found to contain at least nine alpha helices, referred to as helix A through helix I, nine beta sheets, referred to as b1 through b9 (Tabor et al., Phil. Trans. R. Soc. Lond. B340:315 (1993), Mohammadi et al., Cell 8:577 (1996) and Hubbard et al., Nature 372:746 (1994)). The HJ loop is a continuous sequence of about twenty amino acids beginning within the F helix about five amino acids residues from the N-terminus of the F helix and extending about five amino acid residues into the G helix. It is noteworthy that the HJ-loop of the TGF_{β}ILK family of protein kinases contains an insertion of about 12 to 15 extra amino acids as compared to other ser/thr or tyrosine (tyr) protein kinases.

U.S. patent application Ser. No. 09/734,520 discloses a region termed the “A-region”. The “A region” referred to herein is found within the kinase domain of PKs in Subdomain III and Subdomain IV With respect to the amino acid sequence of the prototypical protein kinase PKA-C_{α} the A region can be said to correspond to a contiguous sequence of about eighteen amino acid residues found between about amino acids 92 and 109 of PKA-C_{α}. In some PKs, extra amino acids can be present in this region and the size of the A region can, therefore, include more than 18 amino acids in length.

With respect to the secondary structure of protein kinases, the A region is a contiguous sequence of about five to twenty amino acids beginning at the middle of the _{α}C helix (hereby _{α}C) and ending at the beginning of the b4 beta sheet.

U.S. patent application Ser. No. 09/458,491 discloses a region termed B4-B5 region. The “B4-5 region” referred to herein is found within the kinase domain of PKs in Subdomain IV and the beginning of Subdomain V With respect to the amino acid sequence of the prototypical protein kinase PKA-C_{α}, the B4-5 region can be said to correspond to a contiguous sequence representing the amino acid residues found between about amino acids 106 and 114 of PKA-C_{α}.

In some PKs, extra amino acids might be inserted in this region and the size of the B4-5 region can, therefore, include more than 9 amino acids in length.

A second definition of the B4-5 region of a PK, which is complementary to the definition provided in the preceding paragraph, can be made by reference to the three dimensional structure of the kinase domain of PKs. The kinase domain of PKs has been found to contain at least nine alpha helices, referred to as helix A through helix I and nine beta sheets, referred to as b1 through b9 (Tabor et al., Phil. Trans. R. Soc. Lond, B340:315 (1993), Mohammadi et al., Cell, 86:577 (1996) and Hubbard et al., Nature 32:746 (1994). The B4-5 region is a contiguous sequence of about five to twenty five amino acids beginning at the end of the b4 beta sheet and into the b5 beta sheet.

U.S. patent application Ser. No. 09/161,095 discloses a region termed the “_{α}D region”.

The “_{α}D region” referred to herein is found within the kinase domain of PKs in Subdomain V and the beginning of Subdomain VI. The “_{α}D region” of a PK can be defined by reference to the amino acid sequence of a prototypical protein kinase, for example PKA-C_{α} and can be said to correspond to a contiguous sequence of about twenty amino acid residues found between about amino acid 120 and 139 of PKA-C_{α}.

In relation to the secondary structure of the kinase domain of PKs, the _{α}D region is a contiguous sequence of about fifteen to forty amino acids beginning at the end of the b5 beta sheet and extending through the D helix and the following loop to the beginning of helix E.

The three dimensional structure of a number of kinases has bee determined. A classical view of this structure is given in Knighton et al., Science 253, 407-414 (1991).

### SUMMARY OF THE INVENTION

The present invention is based on the realization that while peptides derived from the above four regions, as disclosed in the above-mentioned applications, can specifically modulate the kinase associated signal transduction (of the kinase from which they were derived) they still feature the inherent disadvantages of peptides: i.e. problems in penetration through cellular membranes, degradation by proteases, short half-life as well as high cost in production. The present invention is based on the realization that it is preferable to use non-peptidic organic molecules for modulation purposes, and that the identification (synthesis or screening) of these molecules may be carried out by using information concerning the three dimensional structure of these regions.

The present invention is further based on the realization that not all moieties of the above four regions are required for the interaction of the kinase with the other cellular components. The present invention thus concerns method for finding the exposed moieties in specific regions of kinases, which regions are involved in the interaction of the kinase with other cellular components and which regions are believed to be specific to the kinase or kinases of the same family, so that the manipulation of the interaction of the kinase through these regions is specific to the kinase or to a kinase of the same family.

The present invention further concerns the atomic coordinates of those specific moieties that are exposed in the above specific regions which are termed hereinafter: as “HJ-loop, the A-region, the B4-B5 region, or the _{α}D-region” in a large number of protein kinases. The determination of the atomic coordinates and the nature of the exposed moieties in each of the above regions of the kinase, enables the realization of the three dimensional structure and basic chemical properties (charged, hydrogen donor/hydrogen acceptor, polar or hydrophobic) of those sites in these regions of the kinase that are capable of interacting with other cellular components. The coordinates' data enables obtaining (by screening or by synthesis) of compounds that either mimic or bind to the specific regions, and by this modulate the interaction of the specific kinase with other cellular components and hence modulate that kinase associate-signal transduction (KAST).

The invention makes use of the fact that only the exposed moieties in those four unique regions (exposure as determined by the accessibility of the moieties to water molecules) are those that interact with the other interacting components leading the way to the modulation of the kinase-associated signal transduction.

These findings lead the way to the design, identification or screening, of non-peptidic compounds that can serve as candidates (i.e. have a high probability of being active) for the modulation the KAST.

By one aspect of the invention the compounds mimic the region, and thus are candidates for mimicking the association of this region, with other “interacting cellular components” (see below). This mimicking may lead the way to inhibition of kinase-associated signal transduction by the interruption of the interaction between the kinase and the interacting components leading to a modulation in the level of the KAST (as determined for example by the change in the level of the phosphorylation of the kinases substrates or by the change in the level of the physiological popery dependent by that specific KAST).

Alternatively the mimicking of the region may cause enhancement of the kinase-associates signal transduction, for example by conferring a conformational change in the interacting component leading to increased KAST. The enhancement of the KAST may also be due to inhibition of cellular interrelations which “turns off” kinase activity, such as by turning off the inhibitory activity of phosphatases that dephosphorylize either the kinase or its substrates, thus prolonging of the kinase enzymatic activity and increasing the overall kinase associated signal transduction.

By another aspect these findings lead the way to the identification, design or screening of compounds that can bind to one of these regions (also termed herein as “docking”). Again the binding may lead to inhibition of the kinase-associated signal transduction by interruption of the interaction between the kinase and the interacting components, due to the fact that the site in the kinase involved with this interaction is masked. Alternatively the binding may lead to a conformational change that activates the kinase, or may lead to a change that interrupts the binding of the kinase to inhibiting components such as phosphatases thus increasing (prolonging) the kinase activity and increasing the overall KAST.

Thus by a first aspect termed “the mimicking aspect” the present invention concerns molecules or molecular complexes that are candidates for mimicking the three-dimensional structure and the basic chemical properties the exposed moieties of any of the HJ-loop, the A-region, the B4-B5 region, or the _{α}D-regions of a specific kinase, and thus are candidates for serving as modulators of that specific kinase-associated signal transduction.(or the signal transduction associated with a kinase of the same family Thus the present invention concerns a compound which is a candidate for serving as a modulator for a kinase associated signal transduction, the compound (or some of its moieties) having similar basic chemical properties and similar atomic coordinates as at least three, preferably at least four most preferably at least five, exposed moieties in any of, an HJ-loop, a-region, B4-B5 region or _{α}D-region of the specific kinase or of a kinase of the same family.

Examples of coordinates of the exposed moieties are given in Table 1 of the attached CD-R.

By a second aspect termed “the binding aspect” the present invention concerns molecules or molecular complexes that are candidates for binding to the exposed moieties in these regions of a specific kinase and thus are candidates for modulation of kinase-associated signal transduction.

Thus the present invention concerns a compound which is a candidate for serving as a modulator of the activity of a specific kinase-associated signal transduction by binding to the kinase, the compound capable of binding to at least three preferably at least four most preferably at least five, exposed moieties, present in an HJ-loop, A-region, B4-B5 region or _{α}D-region of the specific kinase or of a kinase of the same family. Examples of coordinates of the exposed moieties are given in Table 1 of the attached CD-R.

Each of such compound mimicking or binding compounds is a candidate suitable for the modulation of the activity of a signal transduction associated with a specific kinase or a kinase of the same family the specific kinase, the term “family” referring of a small group of kinases that have structural similarity or similarity in the interacting components to which the kinase binds.

It should be noted that each mimicking compound and each binding compound is suitable for the modulation of the activity of that specific kinase which atomic coordinates were used (for its synthesis or screening), or of a kinase of the same family. Thus the compounds of the inventions are candidates for specifically modulating the KAST of a kinase or a family of kinases without affecting the KASTs of other kinases or of kinases of another family.

The invention also provides a hardware-software system comprising machine-readable storage medium which comprises data describing the atomic coordinates and the basic chemical properties (i.e. whether the exposed moieties are C, N, O, NH, OH, OC or S atoms) in the above four regions, obtained from a plurality of kinases, Preferably the above data is grouped according to kinase families.

Such storage medium encoded with this data is capable of being displayed on a computer screen or similar viewing device. The display maybe of a three dimensional graphical representation of the exposed moieties of region of the specific kinases, or a display of the data of the coordinates and the nature of the exposed atoms themselves, for example, in the form of a table. An example of such a table is Table 1 as present on the CD-R.

The invention provides a hardware software system for displaying information of the structure of the exposed moieties of a HJ-loop, A-region, B4-B5 region or _{α}D-region of a plurality of kinases, the system comprising:

(a) a machine readable data storage medium comprising machine-readable data concerning the basic chemical properties of the exposed moieties and the atomic coordinates of exposed moieties of a HJ-loop, A-region, B4-B5 region or _{α}D-region of each kinase of the plurality of kinases;

(b) a software package comprising instructions for processing and displaying said machine readable data;

(c) hardware suitable for reading said data, running said software package, and means for displaying said data.

The present invention also provides methods for designing, evaluating or identifying (for example by screening from a databases of available chemical compounds) compounds that are suitable for serving either as candidate mimic compounds or as candidate binding compounds of a specific kinase (or kinase family) thus serving as modulators of the KAST of that specific kinase or a kinase of the same family.

1.. The present invention further concerns a method for evaluating the potential of a chemical compound to mimic at least three (preferably at least four, most preferably at least five or six) moieties which are exposed in an HJ-loop, A-region, B4-B5 region or _{α}D-region of a specific kinase or of a kinase of the same family, the method comprising:

(a) aligning the catalytic unit of the kinase and the catalytic unit of PKA-c a and finding sequences in the kinase that correspond to the following regions in PKA-ca:: 229 to 248 (HJ-region); 92 to 109 (A-region); 106 to 114 (B4-B5); 120 to 139 (_{α}D region);

(b) determining the three dimensional stricture of each of these regions in the kinase;

(c) determining the atomic coordinates and basic chemical properties of the exposed moieties, defined as those moieties that that are accessible to the water's oxygen atom in each of the regions;

(d) employing computational means to perform a fitting operation between the chemical compound and at least three,(preferably at least four, most preferably at least five or six) of the exposed moieties of (c);

(e) analyzing the results of said fitting operation to quantify the similarity between the chemical compounds and the exposed moieties.

Than according to that method those candidate compounds having a high score of similarity are chosen to give candidate compounds for the modulation of KAST

The same method can be used to evaluate the ability of a compound to bind to the exposed moieties and those compounds having a high binding score are chosen as candidates for the modulation of kinase associated signal transduction.

Said method makes use of the machine readable storage medium of the invention either for the synthesis de novo of compounds, or for the screening (using suitable docking or fitting programs) of molecules which are likely candidates for serving as mimic or of binding compounds. The activity of the candidates as actual KAST modulators has to be verified by using suitable assays as for example stipulated in Example 2 in the “Detailed Description” part below.

Generally the candidate binding or mimicking compounds are contacted with an assay wherein the level of a physiological property that is dependent on that specific KAST is assessed. Examples of such properties are phosphorylation of a substrate of the kinase-associated signal transduction pathway, cellular proliferation, cellular differentiation, cellular elongation, cellular migration, apoptosis, metabolism, and secretion of substances. Those candidate compounds that changed the physiological property as compared to control are chosen as modulators of KAST.

The first step of the method begins by identifying the above four regions in the kinase by aligning the catalytic unit of the kinase and the catalytic unit of PKA-C_{α} and determining the amino acids in the positions corresponding in the alignment to the following: HJ-229 to 248 of PKA-C_{α}. A-region: 92 to 109 of PKA-C_{α}. B4-B5 106 to 114 of PKA-C_{α}. Alpha D: 120 to 139 of PKA-C_{α}.

Than the three dimensional structure of the above four regions as present in the native kinase is determined either by using actual crystallographic data or by using homology modeling.

Than the exposed moieties in the three dimensional structure are determined for example by determining which moieties are accessible to the water's oxygen atom. These steps enable the generation of a set of coordinates of the exposed moieties in each of the regions and the determination of the chemical properties of each of the exposed moieties, to produce a set of data for the kinase The generated set of coordinates and chemical properties is used to virtually evaluate the potential of a chemical compound, for example, present in a database of chemical compounds, to mimic (by utilizing a computational fitting operation) or to bind to (by utilizing a computational docking operation), a region in a specific kinase or a region of a kinase of the same family.

Each compound is given a score, which can be a arbitrary number indicating the quality of the fitting or the binding (docking). For calculation of this score both the geometry of the compound and energy functions (being scoring function and/or potential energy function) are taken into consideration.

Then one selects those compounds that have a good fitting score (for mimicking compounds) or a good docking score (for binding compounds), that can mimic or bind, respectively, at least three, preferably at least four, most preferably at least five or six exposed moieties. The selected compounds are the candidate mimic and candidate binding compounds of the invention.

Then those candidate compounds are assayed in an actual experimental assay and those which change (increase or decrease) the level a physiological property dependent on that specific KAST. Compound which were found to be active in the assay are chosen as modulators of KAST that can be used as drug leads for the development of pharmaceuticals.

The present invention also concerns methods for designing (synthesizing) compounds that mimic the three-dimensional structure and the basic chemical properties of the exposed moieties of the HJ-loop, the A-region, the B4-B5 region, and the _{α}D-region and thus are candidates for modulating KAST. This design may be carried out by using state of the ail rational drug design techniques either in a step-wise manner or by synthesizing of the compound as a whole as will be explained herein bellow. The synthesized compounds 9which may be mimics or binging candidates) are evaluated for their mimicking or binding activities as described above and those compounds with a high mimicking/binding score are chosen as likely candidates for the modulation of KAST.

Of course as described above actual KAST modulating properties have to be verified in an assay that measures the physiological properties that are dependent on that specific KAST.

### GENERAL DESCRIPTION OF INVENTION

The term “region” in the context of the present invention refers to any one of the HJ-loop, the A-region, the B4-B5 region, or the _{α}D-region, within the various protein kinases. The regions of the specific kinase to be determined are defined in reference to specific positions in the prototypical kinase PKA-C_{α} when the catalytic unit of the two are aligned.

The term “PKA-C_{α}” refers to cAMP-dependent protein kinase, alpha-catalytic subunit (PKA C-alpha) accession number P05132 version P05132 GI: 125206 as defined in NCBI.

The positions of the regions in reference to PKA-C_{α}. are as follows:

HJ-229 to 248 of PKA-C_{α}.

A-region: 92 to 109 of PKA-C_{α}.

B4-B5 106 to 114 of PKA-_{α}.

Alpha D: 120 to 139 of PKA-C_{α}.

For determining the beginning and end positions of the regions in the specific kinase, the sequence of the catalytic unit of the specific kinase should be aligned with the sequence of the catalytic unit of PKA-C_{α} in pair-wise or multiple-alignment manner. Alignment may be carried out using any state of the art software such as ClustAl™ (version W or X). Alternatively for producing the alignment it is possible to use tables showing these alignments which are available from various published sources, such as, for example, in the article by Hanks and Quinn in Methods of Enzymology 200: 38-62 (1991) or in the PKR Web Site: WWW.sdsc.edu/kinases. In some kinases extra amino or less amino acids may be present in any of the regions and the size of the region can, therefore, include more or less than amino acids in length than those appearing in PKA-C_{α}, however, the alignment methods (both present in ready table or carried out by known programs) can be carried out even if the size of the region of the kinase and the region of PKA-C_{α} are different. It shall be noted that when the kinase is PKA-C_{α} itself the positions are already given.

A complementary manner for identifying the region, which can help in case the alignment is problematic is by reference to the three-dimensional structure of the kinase as explained in the background section of the specification and as illustrated in reference to FIG. 1, below. In terms of the three dimensional structure of kinases, the catalytic domain of kinases has been found to contain at least nine alpha helices, referred to as helix A through helix I and nine beta sheets, referred to as (b1) through (b9) (Tabor et al., Phil. Trans. R. Soc. Lond. B340:315 (1993), Mohammadi et al., Cell, 86:577 (1996) and Hubbard et al., Nature, 372:746 (1994)). Relationships between the primary structure of a large number of protein kinases and their corresponding three dimensional structure is well known in the art.

The term “modulation of Kinase associated signal transduction (KAST)” refers to modulation in the level of the signal transduction that is associated with the kinase, i.e. a change in the level of the phosphorylation of the kinase's direct or indirect substrates. This term does not necessarily mean that the activity of the kinase per se is changed, but rather that the interaction between the kinase and the interacting cellular component is modulated. This may be for example due to binding (of the modulating compound) to the kinase's substrate at the kinase-binding region, and by this interrupting subsequent kinase binding and hence interrupting phosphorylation of the substrate. Modulation may also be of course by direct effect on the kinase. At times the modulation in the KAST may be determined by the measurement of physiological parameters (such as apoptosis, proliferation, differentiation, metabolism, secretion etc.) controlled by the KAST. Typically modulation refers to increase or decrease in the level of the KAST but this term may also refer to a change in the response of the KAST to an external signal such as change in the response to hormones., growth factors, or stress or to the change in the timing of the activity of the KAST (independently or as a response to an external signal).

The term “exposed moiety” in the context of the present invention refers to the relevant atoms, or to atom-hydrogen group within the region that is accessible to water molecules. The accessibility to water molecules is determined by the accessibility of the moieties in this region to the water's oxygen atom (according to the teaching of Lee et al. J. Mol. Biol. 55,379-400(1971)). The exposed moieties are generally N, C, O, S, NH or OH, CO. It should be noted that the above moieties may be fully or partially exposed.

The term “candidate mimicking compound” in the context of the present invention refers to a non-peptidic molecule (see below), or complex of molecules, that at least some of its moieties have a corresponding (see below) three dimensional structure and similar “basic chemical properties” (see below) to those moieties that are exposed (as determined by accessibility to the water's oxygen atom) in the regions of a specific kinase. The candidate-mimicking compound is predicted to mimic the three-dimensional structure and the basic chemical properties of the exposed moieties in the region of the kinase, and thus is assumed to interact with the interacting components, with which the kinase normally interacts) in a manner similar to the manner the kinase interacts with them. Thus the candidate mimicking compound is a candidate for the modulation of the kinase-associated signal transduction. The modulation may be by way of inhibition or activation or by way in change of the response of the KAST to an external signal.

Those candidate mimicking compounds, which were found by empirical assays to be actual modulators of KAST, may be used as a drug to treat diseases wherein a beneficial effect may be evident as a result of modulation of KAST. It should be emphasized that it is merely predicted, with a high degree of probability ,that the candidate mimicking compounds are capable of modulating KAST but the actual determination of modulation should be empirically tested for each compound. The candidate compounds should feature similar three dimensional structure and similar basic chemical properties to at least three, preferably at least four; most preferably at least five, exposed moieties which are determined by their accessibility to the water's oxygen atom. Examples of coordinates of exposed moieties in the regions in a number of kinases is specified in Table 1 present in the attached CD-R.

The term “similar three dimensional structure” or “corresponding three dimensional structure” means that the mimic compound has atomic coordinates which are distanced from each other in a similar range (up to a difference of 4 ANG) to the distances of the corresponding at least three exposed moieties (which it mimics) when present in the native kinase.

The term “binding compound” in the context of the present invention refers to a non-peptidic molecule that can “associate with” exposed moieties in any of the regions.

The term “associate with” refers to a condition of proximity between a chemical entity or compound, or the association of binding portions of the compound ,with the exposed moieties of the region. The association may be non-covalent, wherein hydrogen bonding or Van der Waals or electrostatic interactions, energetically favor the juxtaposition. Alternatively, the binding may be covalent. Preferably the binding compound is capable of binding to at least three, preferably at least four; most preferably at least five exposed moieties (as determined by their access ability to water's oxygen atoms), an example of the coordinates of exposed moieties specified in Table 1 of the attached CD-R.

The binding can modulate the KAST either by inhibition, for example by blocking the interaction of the kinase with the interacting components, or may activate the KAST for example by causing a conformational change in any of the interacting components. The binding compound should preferably have “complementary chemical properties” (see below) to the exposed moieties in the region. Thus the binding compound is a candidate for binding to the kinase and regulating the KAST. The binding compound may be used as a drug to treat diseases wherein a beneficial effect may be evident as a result of modulation of kinase activity.

The term: “non-peptidic” refers to the fact that the compound (both according to the mimicking and according the binding aspects of the invention) is not exclusively made up of naturally occurring amino acids. However this term those not exclude the possibility that the compound may comprise one or more naturally occurring or synthetic amino acids. However where more than one naturally occurring amino acid is included, no more than three amino acids should be linked directly to each other via peptidic linkages.

The term “basic chemical properties” refers to the fact that the exposed moieties (which are O, N, C, NH, OH, OC or S) have properties which can be defined as “polar” (hydrogen donor or hydrogen acceptor) “charged” (positive or negative) or “hydrophobic”. The candidate-mimicking compound should have the same basic chemical properties in similar positions (as defined by atomic coordinates) as the exposed moieties in the region

The term “complementary chemical properties” refers, in the context of the invention, to those properties of candidate binding compounds that enable their binding to the exposed moieties. For example where the atom of the exposed moiety in the kinase is positively charged in a specific position the candidate binding compound, binding to said exposed moiety, should have a chemical functional group that is negatively charged, and vice versa. Where the exposed moieties in the region are hydrogen acceptor the binding compound should be hydrogen donor and vice versa. It should be noted that there is a possibility that different moieties do not interact directly but rather through an intervening group. In such a case it is possible that both the binding compound and the exposed moiety have for example the same charge. For example two acidic side chains of proteins may share a proton (Floco, J. Mol Biol., 254(1), 96-105,(1995).

The term “interacting cellular components” refers to any cellular agent to which the kinase binds. The agent may be a protein or a non-protein agent. Examples of such components are: the kinase substrate, ATP, GTP, other kinases which phophorylate the kinase, other phosphatases which de-phophorylate the kinase, regulatory units of the kinases and the like.

The term “kinase family” in the context of the present invention refers to grouping of individual protein kinases (PK) into distinct subgroups based on structural and functional properties. Typically phylogenetic trees derived from the alignment of kinase domain amino acids sequences serve as basis for family classification. Families may also or in addition be grouped by overall structural topology, mode of regulation, and substrate specificity.

The term “moiety” in the contact of the present invention refers to an atom or a group of atoms which form together a functional group capable of chemical interaction. Typically the moieties are O, N, C, NH, OH, OC or S.

The term “exposed” in the contest of the invention refers to a moiety that is accessible to the oxygen atom in water as can be determined for example by virtually constructing a hemisphere having the diameter of an oxygen atom, virtually “rolling” the hemisphere on the surfaces of the kinase and determining which moieties are capable of “contacting” the virtual hemisphere. (Lee et al, supra).

The term “physiological property” refers to a characteristic of a biological system that may be measured to give a distinct level comparable to the level of that system without the candidate compound. The system may be a cell-free system (and in that case the property may be for example binding, level of phosphorylation), a cellular system (and in that case the property may be elongation, differentiation, proliferation, metabolism, apoptosis, secretion of substances), an ex vivo tissue (and in that case the physiological property may be growth, sprouting of blood vessels etc.) or experimental animals (and in that case the physiological property may be size of tumor, mortality, secretion of substances, healing from a disease, inflammatory parameters, metabolism etc.). Each physiological property is connected with a specific KAST. For example it is known that the Src-associated signal transduction is connected to proliferation and cancer so the property should be proliferation (cellular) or cancer shrinkage (in vivo). The IRK-associated signal transduction pathway is known to be associated to glucose uptake so the physiological property may be glucose uptake by fat/muscle cells, or sugar levels in an animal.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A-1D is a view of the structure of a proto-typical PKA kinase wherein the four regions are marked in black: FIG. 1A HJ-loop, FIG. 1B-aD region, FIG. 1C A-region, FIG. 1D B4-B5 region.

FIG. 2A shows the formulas of compounds that were obtained by screening virtual data bases and were computationally found to be capable of being binding candidates to, the HJ-loop of Lyn tyrosine kinase. FIG. 2B shows the formulas of compounds that were obtained by screening virtual data bases and were computationally found to be capable of being mimicking candidates of, the HJ-loop of Lyn tyrosine kinase

FIGS. 3A-3G shows proliferation of various cancer cell lines in the presence of varying concentrations of the compounds of the invention: 3A of Du-145 prostate cancer cells with compound Ld in DMSO; 3B and 3D of prostate cancer cell line DU-145 in the presence of Lm09 in DMSO; 3C and 3E od prostate cancer cell line PC-3 with compound Lm09 in DMSO; 3F of pancreatic cancer cell line Panc1 with Lm09 in DMSO; and 3G of colon cancer cell lines HT29 in DMSO

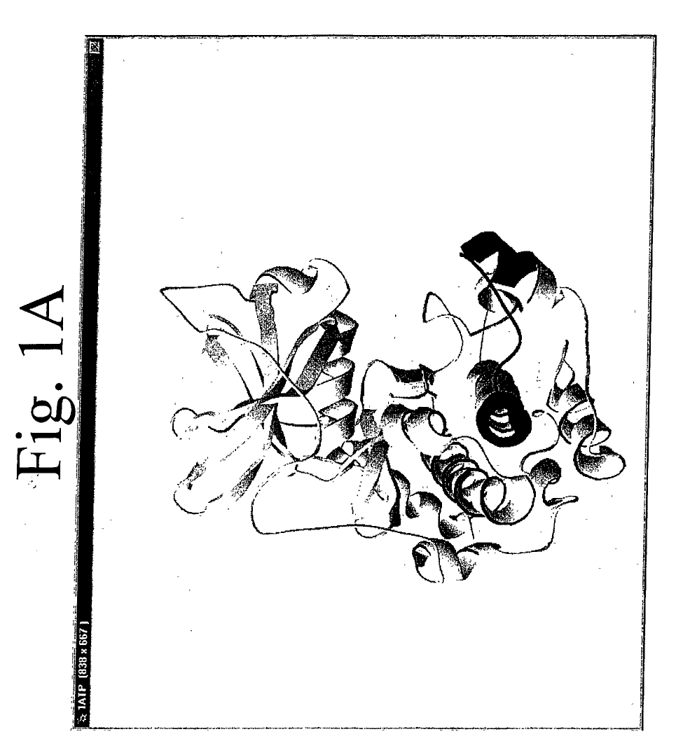

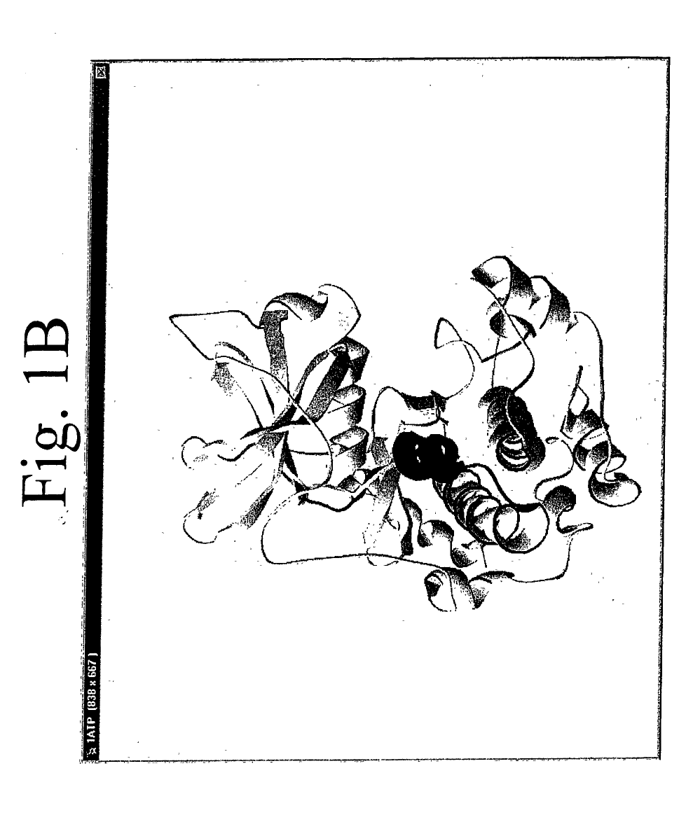

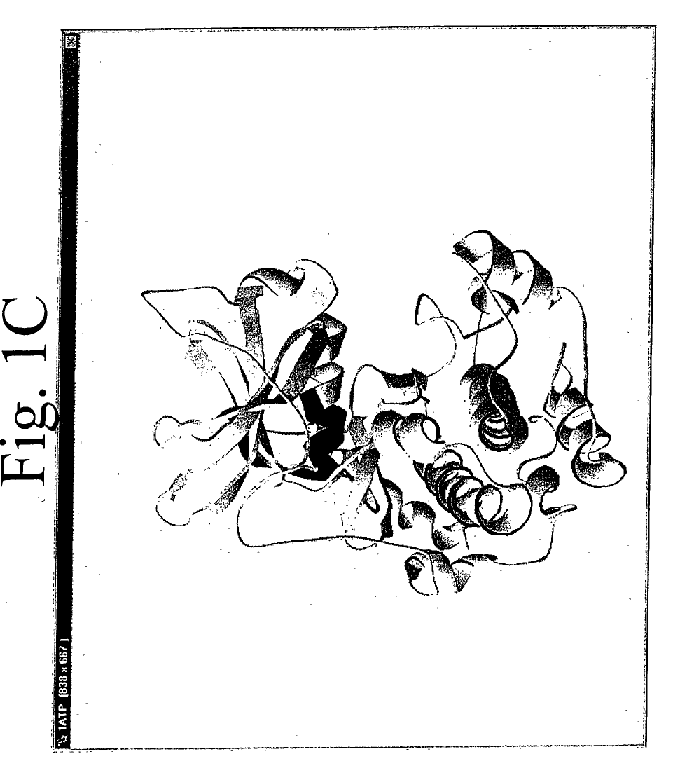

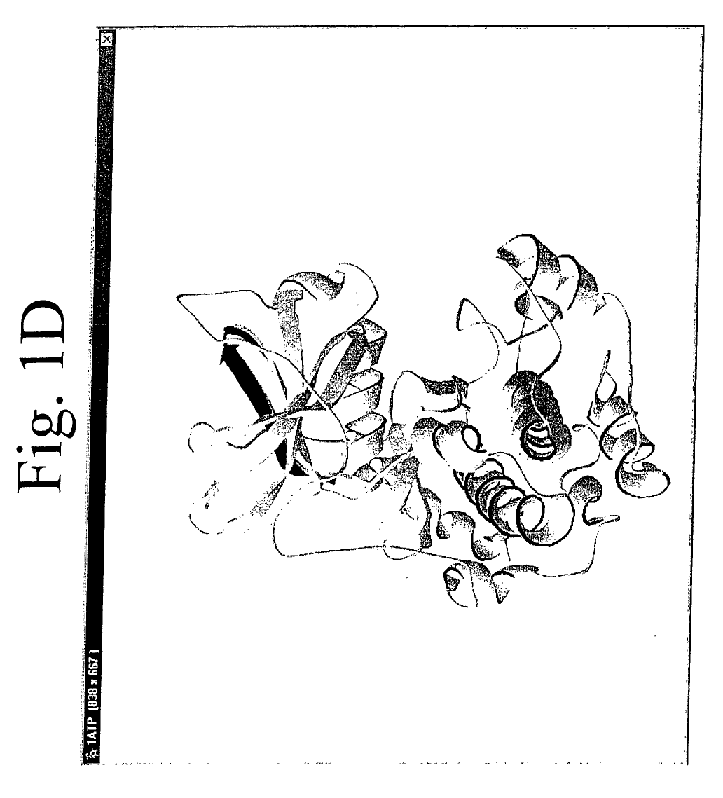

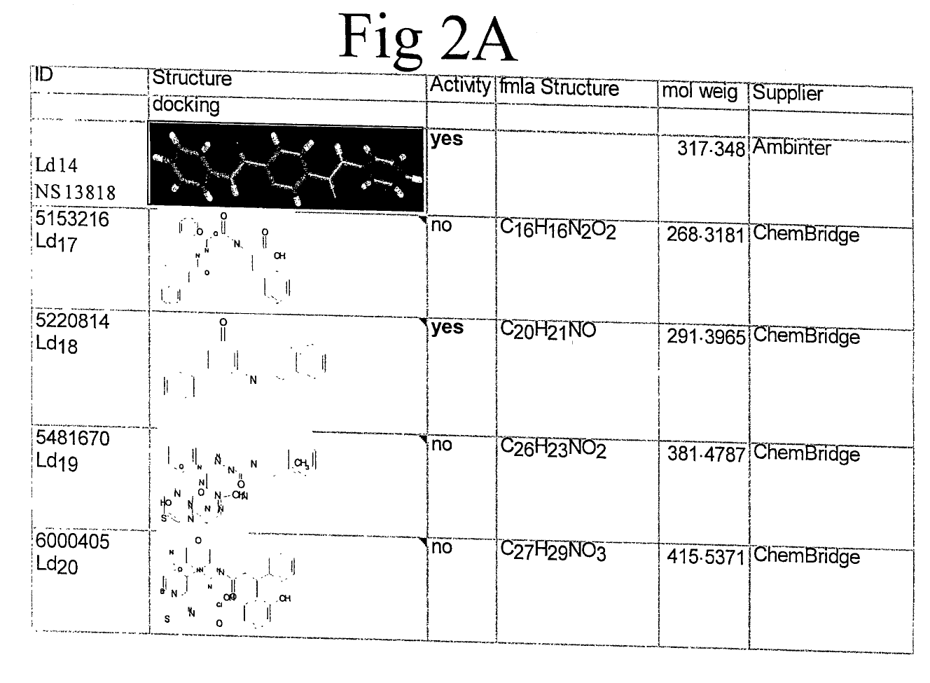

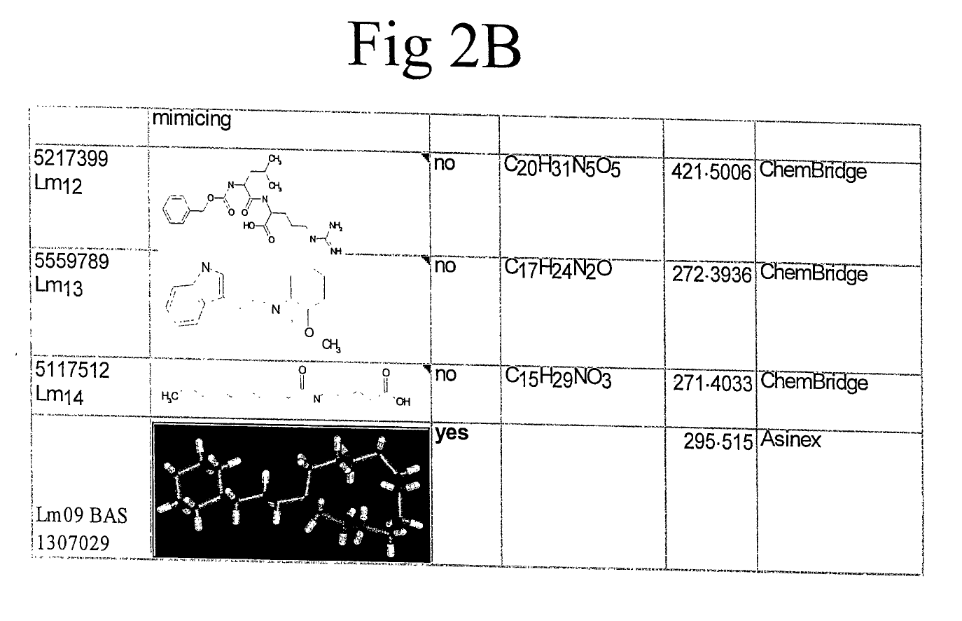

### DETAILED DESCRIPTION OF A PREFERRED EMBODIMENT

### EXAMPLE 1

### Explanation of Table 1 in the CD-R

The attached CD-R forms an integral part of the present invention. The CD-R contains Table 1. The following is an explanation of the Table's structure. Before the atomic coordinates, an identifying indication having the following structure is present: YY_XXXXX. This indication gives the identity of the kinase for which the coordinates are relevant. YY is an arbitrary number indicating the kinase family. XXX is the indication of the specific kinase either by specifying its number in the NCBI (and in that case a number is present) or in the pdb www.rcsb.org/pdb/ (and in that case a combination of digits and numbers is present). For example, where the kinase is cAMP kinase the indication is 25-IBX6. Then the relevant data for each of the regions are specified (HJ-loop, A-region, _{α}D-region, B4-B5 region). The first column gives the running number of the exposed moiety. The second column gives the nature of the moiety itself, and the third, fourth and fifth columns give the x, y, z atomic coordinates, respectively, of the exposed moieties

The moieties are:

| | |
|---|---|
| C | hydrophobic (uncharged) carbon atom |
| OH | hydroxyl group |
| OO | negatively charged oxygen |
| S | Sulfate in cysteine side chains |
| NH | N-atom bound to hydrogen |
| OC | Oxygen bound to carbon |
| CP | positively charged carbon. |

Each of the moieties has basic chemical properties. In the mimicking aspect of the invention, the mimic compound has similar basic chemical properties to at least three, preferably at least four, most preferably at least five or six exposed moieties.

The binding compounds must have complementary chemical properties to at least three or four of the five regions to be able to bind to them. Table 2 below summarizes the nature of the basic chemical properties (for the mimic compound) and the complementary chemical properties (for the binding compound).

TABLE 2

| | | Complementary |
|---|---|---|
| | Basic chemical properties | chemical properties |
| Exposed moiety | (for mimicking candidates) | (for binding candidates) |
| C | Hydrophobic | Hydrophobic |
| OH | *HD/*HA | HD/HA |
| OO | Charged (negative) | Charged (positive) |
| S | HD/HA | HD/HA |
| OC | HA | HD |
| NH | HD/HA | HD/HA |
| CP | Charged (positive) | Charged (negative) |

*HD—hydrogen donor
*HA—hydrogen acceptor

As regards the atomic coordinates, it should be understood that these are merely an average of the multitude of transitions a protein can have in an aqueous medium. Thus, it should be understood that the coordinates serve merely as guidelines, or averages, of the relative distances between the exposed moieties and those moieties are by no means hard-and-fast parameters. Typically, chemical compounds selected (from among a library of potential compounds) as candidates of the invention may differ from these exact coordinates, for example by 2-4 angstroms. It should be noted that many chemical compounds are flexible, and may still be able to form by conformational change a construct that fits or is capable of docking to the relevant region, even if the atomic coordinates of the chemical compound as specified in the data base are merely similar and not identical to those of the invention.

### EXAMPLE 2

### Determination of the Exposed Moieties

The exposed moieties are discovered by drawing the van der Waal's surface of a protein molecule and determines the accessibility of molecules (oxygen) in the solvent (water) is assessed. The full description of the procedure is disclosed in Lee et al, J. Mol. Biol. 55, 379-400 (1971).

### EXAMPLE 3

### Computational Methods For Finding Candidate Mimicking Compounds And Candidate Binding Compounds

3.1 General Considerations

Those of skill in the art understand that a set of coordinates for a protein is a relative set of points that define a shape in three dimensions. Thus, it is possible that an entirely different set of coordinates could define a similar or identical shape. Moreover, slight variations in the individual coordinates will have little effect on overall shape. Therefore as regards the atomic coordinates of the candidate mimic compound or the candidate binding compound, these variations would not be expected to significantly alter the nature of the actual molecule or molecular complex that could be used as the candidate mimic or binding compounds, for modulation of the KAST, in particular, bearing in mind that the compounds may be resilient and may assume a large number of shapes.

The variations in coordinates discussed above may be generated due to mathematical manipulations of the kinase structure coordinates. It should be understood that any set of coordinates that could be mathematically manipulated by rotation or translation (Unitarian transformations) to produce the coordinated given in the present case are also considered as essentially equal coordinates

For the purpose of this invention, any molecule or molecular complex or which serve as the candidate mimicking compound or the candidate binding compound that has a root mean square deviation of less than 2-4 ANG. when superimposed on the relevant atoms (discovered by using the method specified in example 2 above) for example described by structure coordinates listed in Table 1 of the attached CD-R are considered identical. More preferably, the root mean square deviation is less than 2-4.ANG.

The term “root mean square deviation” means the square root of the arithmetic mean of the squares of the deviations from the mean. It is a way to express the deviation or variation from a trend or object.

3.2 Display of Data

In order to use and understand the coordinates, it is sometimes necessary to convert them into a three-dimensional shape. This is achieved through the use of commercially available software that is capable of generating three-dimensional graphical representations of molecules or portions thereof from a set of atomic coordinates.

Therefore, according to another embodiment of this invention there is provided a hardware-software system comprising machine-readable storage medium comprising a data storage material encoded with machine readable data which, when using a machine programmed with instructions for using said data, is capable of displaying a graphical three-dimensional representation of the exposed moieties in any of the regions which the candidate mimicking compound mimics or to which the candidate binding compound binds. The atomic coordinates are generated according to the method of example 2 above. Preferably, the machine-readable storage medium comprises the data specified in Table 1 of the CD-R.

Even more preferred is a machine-readable data storage medium that is capable of displaying a graphical three-dimensional representation of a molecule or molecular complex that is defined by the structure coordinates specified in Table 1, +/−a root mean square deviation from the positions specified in those amino acids of not more than 2-4 ANG

Thus, in accordance with the present invention, the machine-readable storage medium comprises data capable of displaying the three dimensional structure of the exposed moieties of the HJ-loop, the A-region, the B4-B5 region and the _{α}D-region of a plurality of kinases. Such data may be used for a variety of purposes, such as discovery of candidate mimicking or candidate binding compounds that have a high probability of modulating kinase activity and thus can serve as drugs. Various hardwares for reading and displaying such data are well known in the art, and an example of such hardware is specified in U.S. Pat. No. 6,128,582.

3.3 Evaluating or Screening of Existing Compounds

For finding a candidate mimicking compound, the structure encoded by the data may be computationally evaluated for its ability to fit the various protein regions, and such a fit may have a predictive value to estimate the likelihood of a compound for serving as a likely mimicking candidate.

Compounds that have similar three dimensional structure of at least three, preferably at least four, most preferably at least five or six, exposed moieties of the HJ-loop, the A-region, the B4-B5 region and the _{α}D-region, and similar basic chemical properties as the exposed moieties are potential candidates for serving as mimicking compounds. Chemical entities that can bind to at least three, preferably at least four, most preferably at least five or six exposed moieties are potential candidates for serving as binding compounds.

Alternatively, the structure encoded by the data may be displayed in a graphical three-dimensional representation on a computer screen. This allows visual inspection of the structure, as well as visual inspection of various candidate compounds that should have similar structure.

Using databases of coordinates of available chemical compounds (to be screened for the identification of the candidates of the invention) in conjunction with suitable computational techniques may lead to the identification of candidate mimicking or candidate binding compounds.

Various computational analyses are necessary to determine whether a compound or the binding portion thereof is sufficiently similar to all or parts of the exposed moieties of the regions described above. Such analyses may be carried out in current software applications, such as the Molecular Similarity application of QUANTA (Molecular Simulations Inc., San Diego, Calif.) or Unitary™ (Tripos), and as described in the accompanying User's Guide

The Molecular Similarity application permits comparisons between different structures, different conformations of the same structure, and different parts of the same structure. The procedure used in Molecular Similarity to compare structures is divided into four steps: 1) load the structures to be compared (i.e. the virtual structure of the chemical compound in the library and the virtual structure of the three dimensional of the exposed moieties as present in the native kinase for example as determined by the method of Example 2 above) 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein side chain atoms (C, OH, OO, S, OC, NH, CP) for all conserved residues between the two structures being compared. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or binding portion of the molecule that has a root mean square deviation of the relevant moiety (C, OH, OO, S, OC, NH, CP) of less than 2-4 .ANG. When superimposed on the relevant atoms of the exposed moieties as generated by the method of Example 2 (and as stipulated for example by atomic coordinates listed in Table 1 of the CD-R) are considered identical. More preferably, the root mean square deviation is less than 2-4 .ANG

Thus, according to another embodiment, the invention relates to a method for evaluating the potential of a chemical compound to serve as candidate mimicking compounds or candidate binding compounds of the invention.

For evaluating the potential of a chemical compound to serve as mimicking compound this method comprises the steps of: a) employing computational means to perform a fitting operation between the chemical compound and the exposed moieties of any of the HJ-loop, the A-region, the B4-B5 region and the _{α}D-region of a specific kinase; b) analyzing the results of said fitting operation to quantify the similarity between the chemical compound and the exposed moiety in the region; c) a high similarity between the chemical compound and at least three exposed moieties in the region indicating that the compound can serve as a candidate for modulating kinase activity.

Preferably the similarity should be of at least four, most preferably at least five to seven exposed moieties. For giving the score of similarity in the fitting operation, the geometry of the compound should overlap that of the exposed moieties in the region. Preferably the chemical properties should be considered by calculating the energy function (scoring function and/or potential energy function that are approximations of the free energy function).

For evaluating the potential of a chemical compound to serve as a binding compound this method comprises the steps of: a) employing computational means to perform a docking operation between the chemical compound and the exposed moieties of any of the HJ-loop, the A-region, the B4-B5 region and the _{α}D-region of a specific kinase; b) analyzing the results of said docking operation to quantify the association between the chemical compound and the exposed moiety in the region; c) a good association between the chemical compound and at least three (preferably at least four, most preferably at least five or six) exposed moieties in the region indicating that the compound can serve as a candidate for modulating kinase activity. Preferably the association should be of at least four; most preferably at least five to seven exposed moieties.

Again, as indicated above, the geometry of the compound should overlap that of the exposed moieties in the region. Preferably the chemical properties should be considered by calculating the energy function (scoring function and/or potential energy function that are approximations of the free energy function). In addition for binding, the chosen compound should have as small a difference as possible in the energy of their free vs. bound states.

Each product and company, utilizing fitting or docking operations have their own manner of choosing from among all possible chemical compounds. Some simply chose a certain number of compounds (or a certain percentage) having the best score as compared to the others. Other products or systems have their own calibration of a threshold score based on past experience empirically tested

The term “fitting operation” in the context of the invention referrers to a computational method for identifying compounds with a geometry overlapping that of the region of interest (as determined by an overlap of the center of atoms). Preferably the operation also takes into account the energy function of the evaluated compound. The fitting operation is used to evaluate whether a chemical compound can serve as a candidate mimicking compound.

The term “docking operation” in the context of the invention refers to a computational method for identifying compound with complementary geometry and complementary chemical properties to a region of interest. The docking operation is used to evaluate whether a chemical compound can serve as a candidate binding compound.

The term “chemical compound”, as used herein, refers to chemical compounds, as well as some individual moieties (not necessarily continuous) of such compounds.

The above method is described for the evaluation of one chemical compound for its potential for serving as a candidate mimicking or candidate binding compound. For screening purposes this method is simply repeated a plurality of times for each chemical compound present for example in a virtual data base wherein the atomic coordinates and the nature of each chemical compound are given. This can be done for example by using a database comprising the atomic coordinates of a plurality of available chemical properties and sequentially fitting or docking each compound with each of the region of the kinases for example as specified in the CD-R, as will be described in 3.5 below.

3.4 Optimization of the Candidate-Binding Compound

Once a candidate binding compound has been designed or selected by the above methods, the efficiency with which that entity may bind to a specific kinase region or the specific interacting component may be tested and optimized by computational evaluation. For example, an effective binding compound must preferably demonstrate a relatively small difference in energy between its bound and free states of the evaluated compound (i.e., a small deformation energy of binding). Thus, the most efficient binding compounds should preferably be designed with deformation energy of binding of not greater than about 10 kcal/mole, more preferably, not greater than 7 kcal/mole. Binding compounds may interact with the exposed moieties in the region in more than one conformation that is similar in overall binding energy. In those cases, the deformation energy of binding is taken to be the difference between the energy of the free entity and the average energy of the conformations observed when the compound binds to the kinase. An entity designed or selected, as a candidate binding compound may be further computationally optimized so that in its bound state it would preferably lack repulsive electrostatic interaction with the target kinase and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ©1995); AMBER, version 4.1 (P. A. Kollman, University of California at San Francisco, ©1995); QUANTA/CHARMM (Molecular Simulations, Inc., San Diego, Calif. ©1995); Insight II/Discover (Molecular Simulations, Inc., San Diego, Calif. ©1995); DelPhi (Molecular Simulations, Inc., San Diego, Calif. ©1995); and AMSOL (Quantum Chemistry Program Exchange, Indiana University). These programs may be implemented, for instance, using a Silicon Graphics workstation such as an Indigo® with “IMPACT” graphics. Other hardware systems and software packages will be known to those skilled in the art.

3.5 Screening of Databases

Another approach enabled by this invention, is the computational screening of small molecule databases for chemical entities or compounds that can bind in whole, or in part, to the exposed moieties in the region of kinases. In this screening, the quality of association of such entities to the binding site may be judged either by shape complementarily or by estimated interaction energy [E. C. Meng et al., J. Comp. Chem., 13, pp. 505-524 (1992)].

One skilled in the art may use one of several methods to screen chemical compounds or fragments for their ability to have a similar (corresponding) structures (for mimicking purposes) or structures capable of association (for binding purposes) to the exposed moieties of the HJ-loop, the A-region, the B4-B5 region and the _{α}D-region. This process may begin by visual inspection of, for example, of a specific region of one kinase on the computer screen based on the structure coordinates generated by the method specified in Example 2 above and given in Table 1 of the CD-R or other coordinates which define a similar shape generated from the machine-readable storage medium. Selected fragments or chemical compounds may then be positioned in a variety of orientations either for establishing corresponding structures or associated structures, (docked), within that structure defined by the exposed moieties of the region as defined supra. Docking (for binding) and correspondence (for mimicking) may be accomplished using software such as Quanta™, Calayst™,(shape™ in MSI), Insight II™ (MSI), SearchCapture™, Unity™ (Tripos), Sybile™ (Tripose), followed by energy minimization and molecular dynamics with standard molecular mechanics force fields, such as CHARMM™ and AMBER™.

Specialized computer programs may also assist in the process of selecting fragments or chemical compounds suitable for forming candidate binding compounds. These include: 1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK. 2. MCSS (A. Miranker et al., “Functionality Maps of Binding Sites: A Multiple Copy Simultaneous Search Method.” Proteins: Structure, Function and Genetics, 11:29-34 (1991)). MCSS is available from Molecular Simulations, San Diego, Calif. 3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif 4. DOCK (I. D. Kuntz et al., “A Geometric Approach to Macromolecule-Ligand Interactions”, J. Mol. Biol., 161: 269-288 (1982)). DOCK is available from University of California, San Francisco, Calif. Once suitable chemical compound or fragments have been selected, they can be assembled into a single compound or complex. Assembly may be preceded by visual inspection of the relationship of the fragments to each other on the three-dimensional image displayed on a computer screen in relation to the structure coordinates of desired region. This would be followed by manual model building using software such as Quanta™ or Sybyl (Tripos Associates, St. Louis, Mo). Useful programs to aid one of skill in the art in connecting the individual chemical compounds or fragments include 3D Database systems such as ISIS (MDL Information Systems, San Leandro, Calif.). This area is reviewed in Y. C. Martin, “3D Database Searching in Drug Design”, J. Med. Chem., 35:2145-2154 (1992).

3.5 Improving Selection for Candidate Mimicking Compounds Carried Out by Screening

Once a plurality of chemical compounds are identified, by the screening techniques discussed above, as having a good fit with the exposed moieties in the region of the application it is possible to improve the selection (by selecting especially favorable compounds). This is done by determining “forbidden spaces” i.e. spaces that should not be occupied by the atoms of the candidate compound so as to enable access of the compound to either the kinase or the cellular interacting components with which the compound interacts without spatial hindrance. For example it is clear that if, in the native kinase, some of the space surrounding the exposed moieties disclosed by the invention is occupied by the various atoms of the kinase, this occupied space can also be occupied by the candidate mimicking compound's atoms without spatially hindering the interaction of the kinase to the cellular interacting components. Therefore from those chemical molecules that were chosen to fit at least three of the exposed moieties, it is preferable to further choose the compounds whose atoms (not necessarily those important for the fit score) overlap as much as possible the atoms surrounding the specific exposed moiety. This will ensure that the atoms of the mimicking compound do not spatially hinder the interaction of the mimicking compound with the cellular interacting component.

Where the information concerning the crystallographic data of the three dimensional structure of the interaction between the region and the interacting component is available (such as in the case of cAMP dependent kinase which was crystallized with its substrate) it is possible to improve the selection more carefully, by choosing only chemical compounds that have both a good fit and do not have any atoms in the positions the interacting component is known to occupy when interacting with the kinase (the “forbidden spaces” are calculated in accordance with the actual interaction data)

3.6 De Novo Synthesis of Candidate Compounds

Instead of proceeding to build a the mimicking or binding candidate compounds in a step-wise fashion one fragment or chemical compound at a time as described above, such compounds may be designed as a whole or “de novo”.

There are many de novo compound design methods including: various MSI programs 1. LUDI (H. -J. Bohm, “The Computer Program LUDI: A New Method for the De Novo Design of Enzyme Inhibitors”, J. Comp. Aid. Molec. Design, 6:61-78 (1992)). LUDI is available from Molecular Simulations Incorporated, San Diego, Calif 2. LEGEND (Y. Nishibata et al., Tetrahedron, 47:8985 (1991)). LEGEND is available from Molecular Simulations Incorporated, San Diego, Calif 3. LeapFrog (available from Tripos Associates, St. Louis, Mo.). 4. SPROUT (V. Gillet et al, “SPROUT: A Program for Structure Generation)”, J. Comput. Aided Mol. Design, 7:127-153 (1993)). SPROUT is available from the University of Leeds, UK.

Other molecular modeling techniques may also be employed in accordance with this invention [see, e.g., N. C. Cohen et al., “Molecular Modeling Software and Methods for Medicinal Chemistry”, J. Med. Chem., 33:883-894 (1990); see also, M. A. Navia and M. A. Murcko, “The Use of Structural Information in Drug Design”, Current Opinions in Structural Biology, 2:202-210 (1992); L. M. Balbes et al., “A Perspective of Modem Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. ODin. Struct Biology, 4:777-781 (1994)].

### EXAMPLE 4

### Hardware-Software System

The present invention also concerns a hardware-software system. The system comprises a machine-readable data storage medium comprising machine-readable data concerning the nature of the exposed moieties and the atomic coordinates of exposed moieties of a HJ-loop, A-region, B4-B5 region or _{α}D-region. The storage medium may be in any form known in the art such as floppy disks, tapes, zip disks, DVD, CD-R or any other magnetic or electronic storage means.

The system comprises software that is capable of generating three-dimensional graphical structure of molecules or portions thereof, from a set of atomic TM coordinates. Examples are commercially available programs such as spdp Viewer™, Rasmol™, ChemDraw™.

The hardware is typically a computer, such as Silicon Graphic Inc (SGI)®, with display means such as a screen.

The invention also concerns the machine-readable storage medium for use in the above system.

### EXAMPLE 5

### Assays for Determination of Modulation of the Kinase Activity

The candidate mimicking or candidate binding compound of the invention, discovered by the methods of the invention, have to be screened for KAST modulating activity in an experimental assay. Those compounds that are active in the modulation of the KAST have a potential for serving as drugs for the treatment of diseases disorders or pathological conditions wherein a beneficial effect may be evident by the modulation of kinase activity. The assays and tests specified bellow should be specific—i.e. a candidate mimicking or candidate binding compound, that were chosen by screening or synthesizing based on the data of a specific kinase ,should be tested for its activity in the modulation of the specific KAST(of the kinase) or the modulation of KASTs of the same family of kinases.

It can be readily determined whether a candidate compound modulates the activity of a KAST by incubating the candidate compound with cells that have one or more cellular activities controlled by the KAST. The cells are incubated with the candidate compound to produce a test mixture under conditions suitable for assessing the physiological activity controlled by the specific KAST. Example for a KAST dependent activity may be proliferation, apoptosis, elongation, migration, differentiation, metabolism, secretion of substances and the like. The physiological property in the presence of the candidate compound is assessed and compared with a suitable control, e.g., physiological property of the same cells incubated under the same conditions in the absence of the candidate compound. A greater or lesser a property in the test mixture compared with the control indicates that the tested candidate mimicking or binding compound, modulates the KAST and hence the KAST-dependent physiological property.

Suitable cells for the assay include normal cells that express a membrane bound or intracellular kinase, cells which have been genetically engineered to express or overly express kinases, malignant cells expressing a kinases or immortalized cells that express a kinases

Conditions suitable for assessing KAST-dependent cellular activity include conditions suitable for assessing a cellular activity or function under control of the KAST. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., ammo acids, vitamins, growth factors).

In another aspect, the activity of certain kinase (e.g., Akt/PKB, Dudek et al., Science 275:661 (1997)) can be evaluated by growing the cells under serum deprivation conditions. Cells are typically grown in culture in the presence of a serum such as bovine serum, horse serum or fetal calf serum. Many cells, for example, nerve cells such as PC-12 cells, generally do not survive with insufficient serun. The use of insufficient serum to culture cells is referred to as “serum deprivation conditions” and includes, for example, from 0% to about 4% serum. PK activity is determined by the extent to which a peptide or peptide derivative can protect cells, e.g., neuronal cells, from the consequences of serum deprivation. Specific conditions are provided in Dudek et al., and in Example 4 of the application entitled “SHORT PEPTIDES WHICH SELECTIVELY MODULATE INTRACELLULAR SIGNALING” (filed on May 21, 1997, U.S. application Ser. No. 08/861,153), the pertinent teachings of which are incorporated herein by reference.

Generally, malting a quantitative measure of the physiological property that the KAST controls assesses the level of the KAST in the test mixture. The cellular activity can be, for example, cell proliferation. Examples of cells in which proliferation is controlled by KAST include endothelial cells such as bovine aortic cells, mouse MSI cells or mouse SVR cells (see Arbiser et al., Proc. Natl. Acad. Sci. USA 94:861 (1997)), vascular smooth muscle cells, fibroblasts of various tissue origin, and malignant cells of various tissues such as breast cancer, lung cancer, colon cancel; prostate cancer or melanoma. KAST level is assessed by measuring cellular proliferation, for example, by comparing the number of cells present after a given period of time with the number of cells originally present. One example of PKs having to do with cellular proliferation is the receptors of the activin-like kinases (ALKs) super-family

If cells are being used in which the KAST controls cell differentiation (e.g., PC-12 cells transfected with c-Src, see Alema et al, Nature 316:557 (1985)), activity is assessed by measuring the degree of differentiation. Activity can be assessed by measuring the degree to which neurites are extended and the degree to which markers of neuronal differentiation are expressed in PC-12 cells transfected with c-Src; see Alema et al., and the degree to which the formation of mesoderm in developing Xenopus embroya cells is induced; see Burgess and Maciag, Ann. Rev. Biochem. 58:575 (1989) and Dionne et al., WO 92/00999. Activity can also be assessed by the extent to which gene expression; cell morphology or cellular phenotype is altered (e.g., the degree to which cell shape is altered or the degree to which the cells assume a spindle-like structure). One example of a change in cellular morphology is reported in the application entitled “SHORT PEPTIDES WHICH SELECTIVELY MODULATE INTRACELLULAR SIGNALING” (filed on May 21, 1997, U.S. application Ser. No. 08/861,153), which discloses that certain peptide derivatives of the HJ loop of protein tyrosine kinases can cause vascular smooth muscle cells to become elongated and assume a spindle-like shape.

It is to be understood that the assay described hereinabove for determining whether a candidate mimic compound or candidate binding compound modulates a cellular activity or function under the control of a KAST can be performed with cells other than those specifically described herein. KASTs not yet discovered or KASTs whose function is not yet known can also be used in this assay, once it has been determined which cellular functions or activities they control.

Where the substrates of the kinases are known, it is possible to assess the KAST level and the changes in this level as compared to control, by determination of the level of phosphorylation of the substrate protein. Cells known to express the kinase are incubated with a candidate mimicking or binding compound suspected of modulating the kinase activity. Then the cells are lysed, the protein content of the cells is obtained and separated on a SDS-PAGE. Kinase substrates can be identified by use of suitable molecular weight markers, or by using suitable antibodies. The level of phosphorylation of the substrate can be determined by using anti-phosphotyrosine antibodies, either conjugated to a suitable label or further reacted with a label-bearing antibody see Fujimoto et al., Immunity, 13: 47-57 (2000).

Alternatively phosphorylation may be determined in a cell-free system by incubating protein kinase, its substrate and a candidate for modulating kinase activity in the presence of ATP under conditions enabling phosphorylation. The proteins are then subjected to SDS-PAGE, transferred to nitrocellulose followed by immunoblotting by anti-phosphotyrosine antibody. Alternatively it is possible to use [γ-³²P] ATP and quantify the amount of radioactivity in cooperated in the substrate See Fujimoto el al., The J. of Immunol. 7088-7094 (1999).

Other assays for testing kinase-modulating activities may be found in U.S. Pat. No. 6,174,993, WO98/53051 (corresponding to pending U.S. application Ser. No. 08/861,153), WO 00/118895 (corresponding to pending U.S. 09/161,094), U.S. pending application Ser. No. 09/458,491 and U.S. 09/734,520 all incorporated herein by reference.

### EXAMPLE 6

### Screening for Candidate Mimicking Compounds and Candidate Binding Compounds of Lyn Tyrosine Kinase

Lyn tyrosine kinase sequence were obtained from NCBI and the HJ-loop of this kinase was determined, by alignment with PKA C alpha to be in positions 434-458 of the Lyn.

The three dimensional structure of the Lyn was determined using SPDB homology modeling, and the exposed moieties were determined using the method described in example 2 above.

Based on several SAR studies the following groups of exposed amino acids were considered important and their coordinates were used for screening virtal data bases:

1. Ile208 Arg213 Thr214 (IRT)

2. Arg213 Thr214 Asn215 (RTN)

3. Tyr205 Ile208 Arg213 (YIR)

4. Lys207 Ile208 Arg213 (KIR)

5. Ile208 Thr214 Asn215 (ITN)

6. Ile208 Asn215 (IN)

7. Ile208 Arg213 Asn215 (IRN)

Than a virtual library of about several hundreds of thousands of molecules was screened for compounds which are capable of binding or docking (virtually) to the exposed moieties of the HJ-loop of the Lyn kinase according to any one of the seven options mentioned above.

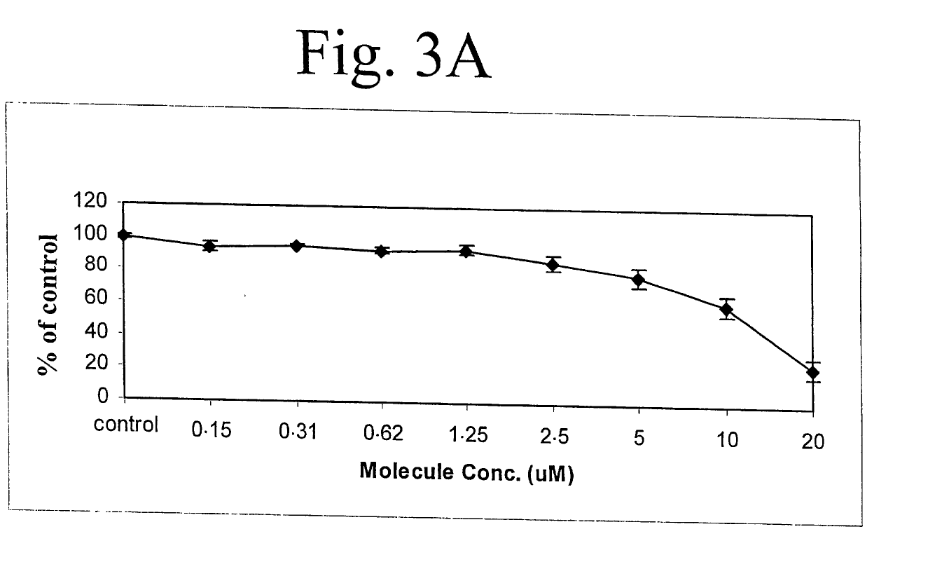

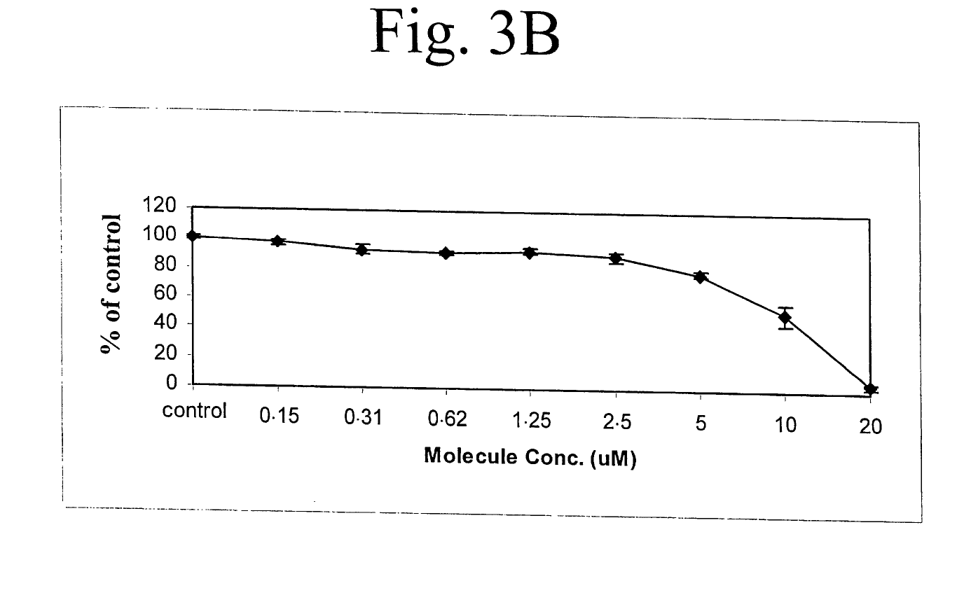

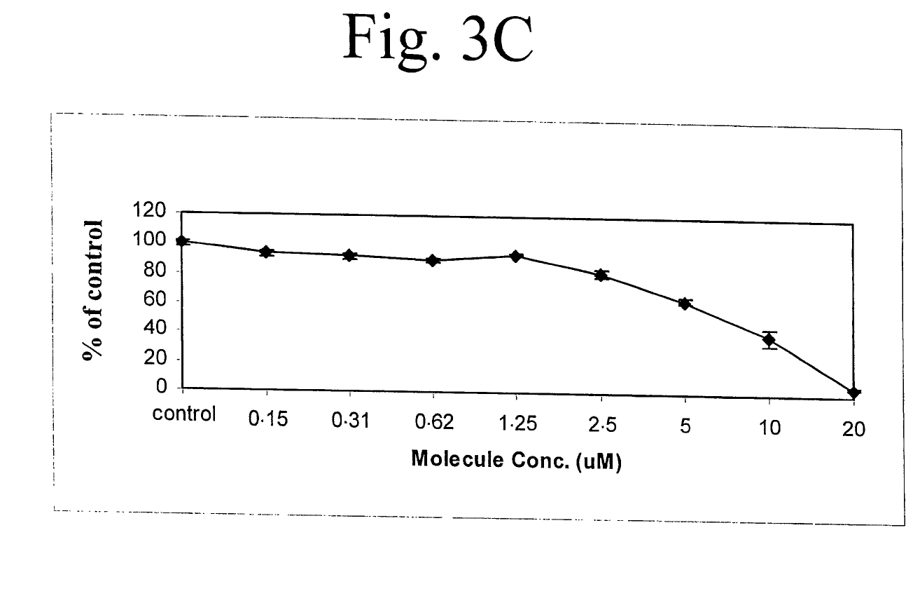

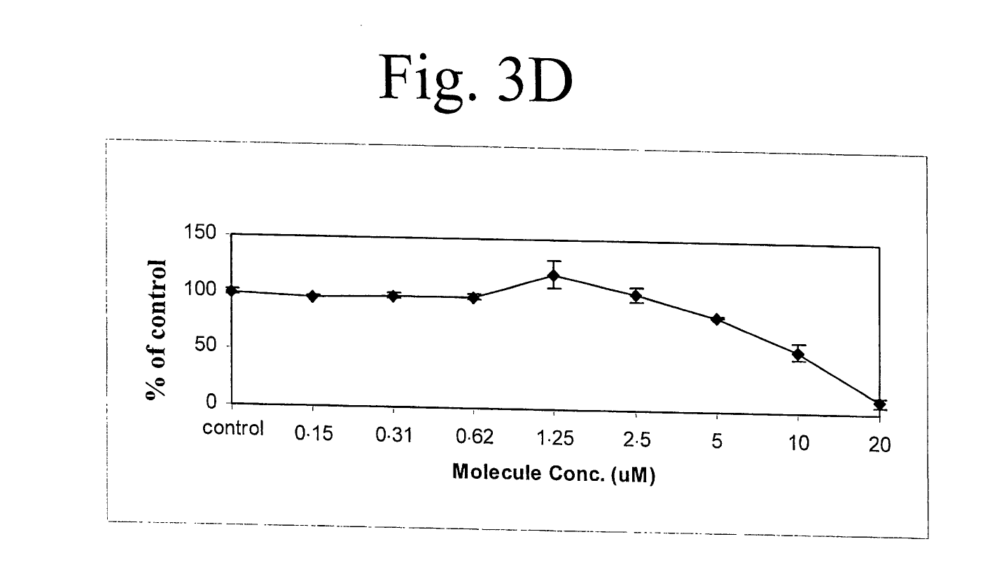

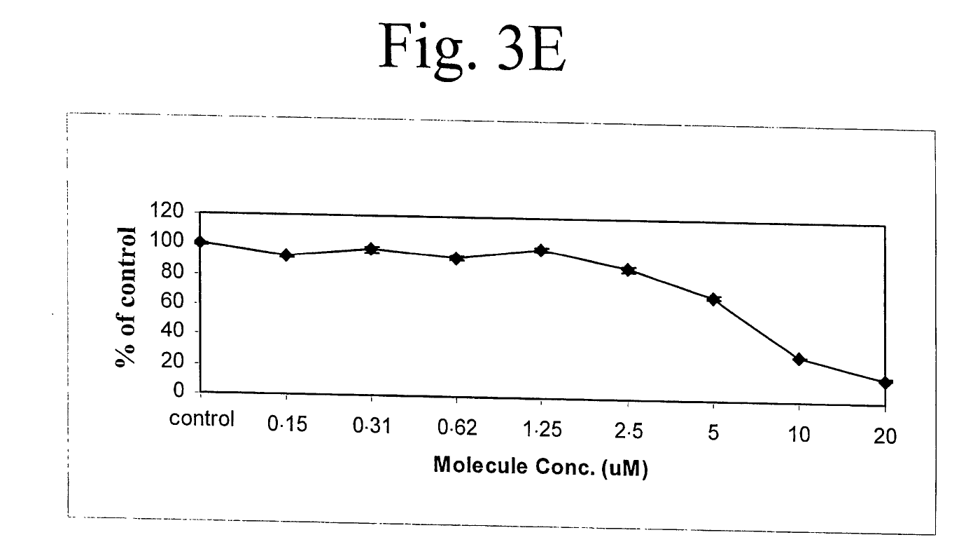

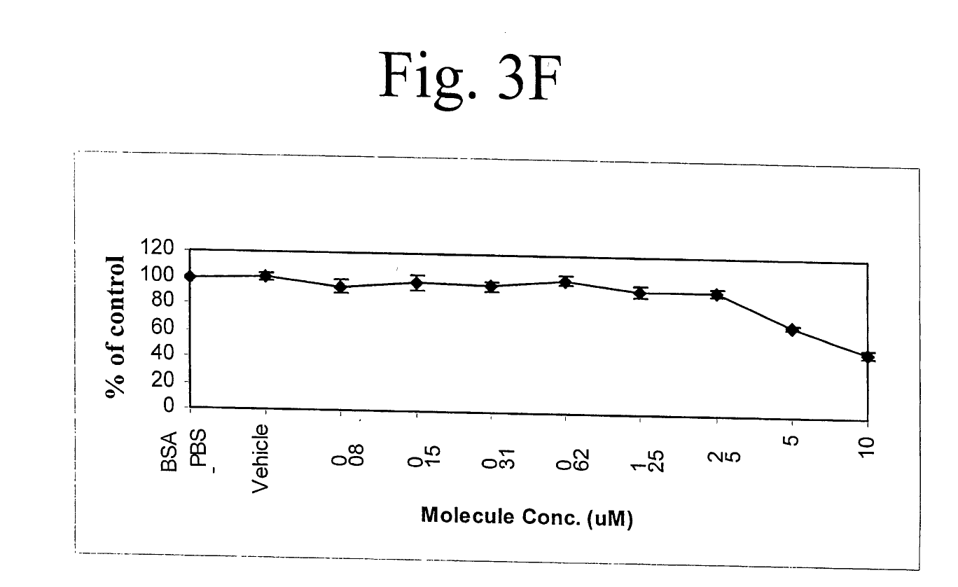

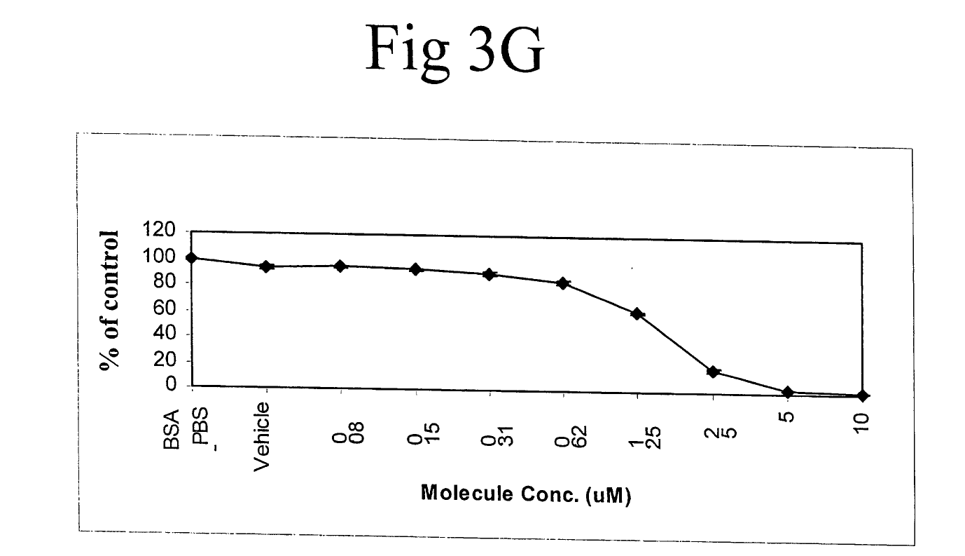

The formulas of the candidate mimicking discovered as above and the candidate binding compounds discovered as above are given in FIG. 3A and candidate mimicking compounds are marked as FIG. 3B. Candidate docking molecules are marked as Lk, and candidate mimicking molecules are marked as Lm.

### EXAMPLE 6

### Screening for Active Modulators of Kinase-Associated Signal Transduction, from the Candidate Compounds

All the compounds of FIG. 3 were screened for inhibition of proliferation of cancer cells, as the proliferation of these cells is dependent on Lyn-associated signal transduction.

DU145, PC-3 prostate cancer cell lines, Panc-1 (pancreas cancer) and HT29 (colon cancer) were obtained from the American Type Culture Collection. These cell lines were grown in RPMI 1640 medium supplemented with penicillin (100 U/ml), streptomycin (100 μg/ml), glutamine (2 mM) and 10% endotoxin free bovine cell serum (Hyclone).

A suspension of the cells at 2×10⁴ cells/ml was prepared in the above described culture mediums and distributed 0.180 ml per well (about 4000 cells/well) in the wells of 96 well, flat bottom, tissue culture microtiter plates.

A series of compounds stock solutions were prepared by diluting a 10 mM solution of the tested compound in 100% DMSO with phosphate buffered saline (PBS) containing 0.1% bovine serum albumin (BSA) to a concentration of 400 μM. These solutions were labeled DMSO. In many instances, 40 μl of the 10 compound in DMSO solution was mixed with 160 μl of 2M NH₄HCO₃ and heated for 40 minutes at 100° C. The resultant solution was then diluted to 400 μM in PBS containing 0.1% BSA. These compounds stock solutions were labeled “tbi”. The concentration of compound in each stock solution was adjusted to nine times the desired concentration of the compound in the assay mixture. 0.020 ml of each compound stock solution was added to the corresponding wells about 2 hours after cell addition, with six replicates for each concentration. In addition, PBS containing 0.1% BSA solution with no added compound was used as a control. The plates were incubated for 72-80 hours at 37° C. in a 10% CO₂ humidified incubator. This formulation was termed “tbi”, and served as a vehicle and as control.

The plates were labeled and the medium discarded. The wells were fixed with 4% formaldehyde PBS (PBS buffered with 10% formalin from Fisher Scientific; Catalog No. HC200-1) (0.2 ml/well) for at least 30 minutes. The wells were washed one time with borate buffer (0.2 ml/well) (0.1 M, pH 8.5). Freshly filtered 1% methylene blue solution (0.60 ml/well) was then added to the wells and incubated for 10 minutes at room temperature. The wells were then washed five times with tap water, after which the wells were dried completely. 0.20 ml/well of 0.1 N HCl was added to extract the color. After overnight extraction, the O.D. was read at 630 nm to determine the number of cells per well. The procedure for counting cells is described in greater detail in Oliver et al. J. Cell Sci., 92: 513 (1989), the teachings of which are incorporated herein by reference.

By this method the compounds of FIG. 3 were screened and mimicking compound Lm09 and binding compound Lk14 were found to be able to inhibit the cells' proliferation of several cancer cell lines in a dose dependent manner as can be seen in FIGS. 3A-3G (wherein 3A of Du-145 prostate cancer cells with compound Ld in DMSO; 3B and 3D of prostate cancer cell line DU-145 in the presence of Lm09 in DMSO; 3C and 3E od prostate cancer cell line PC-3 with compound Lm09 in DMSO; 3F of pancreatic cancer cell line Panc1 with Lm09 in DMSO; and 3G of colon cancer cell lines HT29 in DMSO) indicating that those compounds can alter a KAST-dependent physiological property (proliferation) and thus are modulators of KAST.

1. A compound, which is a candidate for serving as a modulator of a specific kinase-associated signal transduction (KAST), the compound having similar basic chemical properties and similar atomic coordinates as at least three exposed moieties, of an HJ-loop, A-region, B4-B5 region or _{α}D-region of the specific kinase or of a kinase of the same family as the specific kinase.

2. A compound according to claim 1 wherein the exposed moieties are accessible to the water's oxygen atom.

3. A compound according to claim 1 wherein the exposed moieties' coordinates and basic chemical properties are as depicted in Table 1 of the attached CD-R.

4. A compound according to claim 3 having similar basic chemical properties and similar atomic coordinates of at least four exposed moieties, as specified in Table 1, of an HJ-loop, A-region, B4-B5 region of _{α}D-region of the specific kinase or of a kinase of the same family.

5. A compound according to claim 4 having similar basic chemical properties and similar atomic coordinates as at least five exposed moieties, as specified in Table 1, of an HJ-loop, A-region, B4-B5 region or _{α}D-region of the specific kinase or of a kinase of the same family.

6. A compound, which is a candidate for serving as a modulator of a specific kinase-associated signal transduction by binding to the kinase, the compound being capable of binding to at least three exposed moieties, of an HJ-loop, A-region, B4-B5 region or _{α}D-region of the specific kinase or of a kinase of the same family.

7. A compound according to claim 6 wherein the exposed moieties are accessible to the water's oxygen atom.

8. A compound according to claim 7 wherein the exposed moieties' coordinates and basic chemical properties are as depicted in Table 1 of the attached CD-R.

9. A compound according to claim 8, being capable of binding to at least four exposed moieties, as specified in Table 1, of an HJ-loop, A-region, B4-B5 region or _{α}D-region of the specific kinase or of a kinase of the same family.

10. A compound according to claim 9, being capable of binding to at least five exposed moieties, as specified in Table 1, of an HJ-loop, A-region, B4-B5 region or _{α}D-region of the specific kinase or of a kinase of the same family.

11. A method for evaluating the potential of a chemical compound to mimic at least three (preferably at least four, most preferably at least five or six) moieties which are exposed in an HJ-loop, A-region, B4-B5 region or _{α}D-region of a specific kinase or of a kinase of the same family, the method comprising:
a) aligning the catalytic unit of the kinase and the catalytic unit of PKA-c_{α} and finding sequences in the kinase that correspond to the following regions in PKA-c_{α:}: 229 to 248 (HJ-region); 92 to 109 (A-region);. 106 to 114 (B4-B5); 120 to 139 (_{α}D region);
b) determining the three dimensional stricture of each of these regions in the kinase;
c) determining the atomic coordinates and basic chemical properties of the exposed moieties, defined as those moieties that that are accessible to the water's oxygen atom in each of the regions;
d) employing computational means to perform a fitting operation between the chemical compound and at least three,(preferably at least four; most preferably at least five or six) of the exposed moieties of (c);
e) analyzing the results of said fitting operation to quantify the similarity between the chemical compounds and the exposed moieties.

12. A method according to claim 11 wherein the fitting operation is performed with the atomic coordinates of the exposed moieties as specified in Table 1 of the CD-R.

13. A method for screening for chemical compounds which are candidates for mimicking the exposed moieties of a HJ-loop, A-region, B4-B5 region or _{α}D-region of a specific kinase or of a kinase of the same family, and thus a serving as candidates for modulating the signal transduction associated with that kinase the method comprising:
a) evaluating the potential of the chemical compound to mimic the exposed moieties an HJ-loop, A-region, B4-B5 region or _{α}D-region of the specific kinase or of a kinase of the same family, by the method of claims 11 or 12, to quantify the similarity of the compound to the exposed moieties in the region to the region;
b) selecting those chemical compounds that have high similarity with at least three exposed moieties; said compounds being candidates for modulating kinase activity.

14. A method for selecting modulators of kinase activity of a specific kinase or of a kinase of the same family as the specific kinase, the method comprising:.
a) contacting a candidate compound for modulating kinase-associated signal transduction, produced by the method of claim 13 with a biological assay for assessing the level of a physiological property dependent on the KAST, under conditions suitable for the assessment of said physiological property;
b) assessing the level of the physiological property;
c) comparing the level of the property obtained in (b) with the level of property obtained in the same biological assay under the same conditions but in the absence of the candidate compound, a significant difference in the level of the property between the two indicating that the candidate compound is a modulator of kinase-associated signal transduction.

15. A method according to claim 14 wherein the physiological property is selected from: phosphorylation of a substrate of the kinase-associated signal transduction pathway, cellular proliferation, cellular differentiation, cellular elongation, cellular migration, apoptosis, metabolism, and secretion of substances.

16. A method for synthesizing a compound which is a candidate for serving as a modulator of kinase associated signal transduction of a specific kinase or of a kinase of the same family, the method comprising:
synthesizing a compound so that it has a high f similarity with at lease three (preferably at least four, most preferably at least five or six) exposed moieties of an HJ-loop, A-region, B4-B5 region or _{α}D-region of the specific kinase or of a kinase of the same family, said similarity is evaluated by the method of any one of claims 11 or 12.

17. A method for evaluating the potential of a chemical compound to bind to the exposed moieties of an HJ-loop, A-region, B4-B5 region or _{α}D-region of a specific kinase or of a kinase of the same family, the method comprising:
a) aligning the catalytic unit of the kinase and the catalytic unit of PKA-c_{α} and finding sequences in the kinase that correspond to the following regions in PKA-c_{α:}: 229 to 248 (HJ-region); 92 to 109 (A-region); 106 to 114 (B4-B5); 120 to 139 (_{α}D region);
b) determining the three dimensional stricture of each of these regions in the kinase;
c) determining the atomic coordinates and basic chemical properties of the exposed moieties, defined as those moieties that that are accessible to the water's oxygen atom in each of the regions;
d) employing computational means to perform a docking operation between the chemical compound and at least three (preferably at least four, most preferably at least five or six) exposed moieties of the HJ-loop, A-region, B4-B5 region or _{α}D-region of the specific kinase or of a kinase of the same family;
e) analyzing the results of said docking operation to quantify the binding between the chemical compounds and the exposed moieties.

18. A method according to claim 17, wherein the docking operation is performed with the atomic coordinates of the exposed moieties as specified in Table 1 of the CD-R.

19. A method for screening for a chemical compounds that are candidates for binding to at least three (preferably at least four, most preferably at least five or six) exposed moieties of a HJ-loop, A-region, B4-B5 region or _{α}D-region of a specific kinase or of a kinase of the same family, and thus a serving as a candidate for modulating kinase-associated signal transduction, the method comprising:
(i) evaluating the potential of the chemical compound to bind with the exposed moieties of an HJ-loop, A-region, B4-B5 region or _{α}D-region of the specific kinase or of a kinase of the same family, by the method of any one of claims 17 or 18 to quantify the binding between the chemical compound and the exposed moieties to give a binding score;
(ii) selecting those compounds which have a high score of binding to at least three (preferably at least four, most preferably at least five or six) exposed moieties; said compounds being candidates for modulating kinase-associated signal transduction.

20. A method for selecting modulators of kinase activity of a specific kinase or of a kinase of the same family as the specific kinase, the method comprising:.
(i) contacting a candidate compound, selected by the method of claim 19 with a biological assay for assessing a physiological property dependent on the KAST, under conditions suitable for assessing said physiological property;
(ii) assessing the level of the KAST dependent physiological property;
(iii) comparing the level of the physiological property obtained in (b) with the level of the property obtained in the same biological assay under the same conditions but in the absence of the candidate, a significant difference in the level of the property between the two signifying that the candidate compound is a modulator of kinase-associated signal transduction.

21. A method according to claim 20 wherein the physiological property is selected from: phosphorylation of a substrate of the kinase-associated signal transduction pathway, cellular proliferation, cellular differentiation, cellular elongation, cellular migration, apoptosis, metabolism, and secretion of substances.

22. A method for synthesizing a compound which is a candidate for serving as a modulator of kinase-associated signal transduction of a specific kinase or of a kinase of the same family, the method comprising:
synthesizing a compound so that it has a high score of binding with at least three (preferably at least four, most preferably at least five or six) exposed moieties of a HJ-loop, A-region, B4-B5 region or _{α}D-region, of the specific kinase or of a kinase of the same family, said score of binding is evaluated by the method of claims 17 or 18.

23. A candidate compound for modulating kinase activity produced by the methods of claim 13.

24. A candidate compound for modulating kinase activity produced the method of claim claim 19.

25. A modulator of kinase activity produced by the method of claim 14.

26. A modulator of kinase activity produced by the method of claim 16.

27. A hardware-software system for displaying information of the structure of the exposed moieties of a HJ-loop, A-region, B4-B5 region or _{α}D-region of a plurality of kinases, the system comprising:
a) a machine readable data storage medium comprising machine-readable data concerning the basic chemical properties of the exposed moieties and the atomic coordinates of exposed moieties of a HJ-loop, A-region, B4-B5 region or _{α}D-region;
b) a software package comprising instructions for processing and displaying said machine readable data;
c) hardware suitable for reading said data, running said software package, and means for displaying said data.

28. A system according to claim 27 wherein the data comprises Table 1 present on the CD-R.

29. A system according to claim 27 wherein the display is a three dimensional representation.

30. A machine-readable data storage medium for use in the system of claim 27.

31. A storage medium according to claim 30 comprising Table 1 of CD-R.
